(19) United States
(12) Patent Application Publication
(10) Pub. No.: US 2009/0131918 A1
(43) Pub. Date: May 21, 2009

(54) DEVICE AND METHOD FOR THE CONTROLLED RELEASE OF A PREDEFINED QUANTITY OF A SUBSTANCE

(75) Inventors: Franciscus Paulus Maria Budzellar, Eindhoven (NL); Mark Thomas Johnson, Veldhoven (NL); Marc Wilhelmus Gijsbert Ponjee, Tilburg (NL); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/299,128

(22) PCT Filed: Apr. 24, 2007
(86) PCT No.: PCT/IB07/51501

§ 371 Date: Oct. 31, 2008

(30) Foreign Application Priority Data
May 5, 2006 (EP) 06113537.2

(51) Int. Cl. A61K 9/22

(52) U.S. Cl. 604/890.1

(57) ABSTRACT

This invention provides a device for the controlled release of a predefined quantity of a substance and a method of controllably releasing a predefined quantity of a substance from a compartment (20). The device comprises a first number of compartments in a substrate (11), each compartment being closed by at least one release mechanism (30). The device further comprises a second number of actuating contacts (40) for the release of the substance from one compartment of the first number of compartments by means of applying an actuation signal between at least a first actuation contact and a second actuation contact of the second number of actuation contacts, wherein the first number exceeds the second number, and each of the actuation contacts comprises at least one conductor path in or on the substrate forelectrically connecting at least one compartment, and the actuation contacts form a mesh-like structure in or on the substrate.

The present invention relates to a device for the controlled release of a predefined quantity of a substance. The present invention further relates to a method for controllably releasing a predefined quantity of a substance from a compartment.

Accurate delivery of small, precise quantities of one or more chemicals into a carrier fluid are of great importance in many different fields of science and industry. Examples in medicine include the delivery of drugs to patients by means of intravenous methods, pulmonary or inhalation methods or by the release of drugs from vascular stent devices. Examples in diagnostics include releasing reactions in fluids to conduct a DNA or genetic analysis, combinatorial chemistry, or the detection of a specific molecule in an environmental sample. Other applications involving the delivery of chemicals into a carrier fluid include the release of fragrances and therapeutic aromas from devices into air and the release of flavouring agents into a liquid to produce beverage products.

Devices for the controlled release of a predefined quantity of a substance are generally known. For example, US patent application US 2004/0034332 A1 discloses an implantable device for controlled delivery of a drug, the device including a microchip having reservoirs containing the molecules to be released. The microchip device includes a substrate, at least two reservoirs in the substrate containing the molecules to be released and a reservoir cap positioned on or within a portion of the reservoir and over the molecules, so that the molecules are controllably released from the device by diffusion through, or upon disintegration or rupture of, the reservoir caps. Each of the reservoirs of a single microchip may contain different molecules which can be released independently. One drawback of the known device is that each reservoir is directly contacted to an electrode which is used to electrically break the seal layer or the cap by applying a current and to release the drug. A weakness of the prior art system is that one external electrical connection is required for each compartment or for each reservoir from which the drug is to be released. This strongly limits the number of compartments which can be realised on a single device, as the space required for all the electrical connections becomes prohibitive.

It is therefore an object of the present invention to provide a device for the controlled release of a predefined quantity of a substance, that has an increased number of reservoirs or compartments without the need to provide one or more external electrical connections for each compartment to be controlled independently.

The above objective is accomplished by a device and a method for the controlled release of a predefined quantity of a substance according to the present invention, the device comprising, according to a first embodiment of the invention, a first number of compartments in a substrate, each compartment being closed by at least one release mechanism, and the device further comprising a second number of actuating contacts for the release of the substance from one compartment of the first number of compartments by applying an actuation signal between at least a first actuation contact and a second actuation contact of the second number of actuation contacts, wherein the first number exceeds the second number, and each of the actuation contacts comprises at least one conductor path in or on the substrate for electrically connecting at least one compartment, and the actuation contacts form a mesh-like structure in or on the substrate.

The above objective is accomplished by a device and a method for the controlled release of a predefined quantity of a substance according to the present invention, the device comprising, according to a second embodiment of the invention, a first number of compartments in a substrate, each compartment being closed by at least one release mechanism, and the device further comprising a second number of actuating contacts for the release of the substance from one compartment of the first number of compartments by applying an actuation signal between at least a first actuation contact and a second actuation contact of the second number of actuation contacts, wherein the first number exceeds the second number, and each of the actuation contacts comprises at least one conductor path in or on the substrate for electrically connecting at least one compartment, and the one compartment is actuated dependent upon the actuation signal between the first actuation contact and the second actuation contact.

An advantage of the apparatus according to both embodiments of the invention is that it is possible to realize a system for controlled substance or drug delivery based upon a multiplicity of individual drug release compartments, where the number of compartments is very high compared to the number of actuating contacts and the control requirements as well as the manufacturing requirements, and consequently the cost of the device, are reduced. According to the prior art, the number of compartments is strongly limited by the need to contact each compartment individually by a connecting line, or alternatively the costs in terms of control logic and manufacturing costs are comparatively high. The mesh-like arrangement or structure of the actuation contacts hereinafter referred to is for an arrangement wherein at least potentially one release mechanism is provided between each of the actuation contacts.

A further advantage of both embodiments of the present invention is that applications such as, for example, external drug delivery systems (patches), implantable drug delivery systems or oral drug delivery systems (e-pill) are made possible. A drug delivery system according to the present invention may be applied for delivery of a single drug but can be advantageously applied to a system where several different drugs are applied from the same arrangement of compartments or the same device.

Furthermore, it is advantageous that it is possible, according to all embodiments of the present invention, to verify whether the release mechanism intended to be activated is indeed activated, i.e. opened for the release of the substance. In the case of membranes or closure caps as release mechanism, this opening or releasing is also called “blowing” of the membrane or closure cap.

According to the first embodiment of the invention, preferably the first number exceeds one quarter of the square of the second number, and preferably the first number is approximately given by one half of the square of the second number less one half of the second number. As a result, a large first number of compartments can be addressed, requiring only a relatively small second number of actuation contacts. Especially, it is thereby possible to provide a larger first number of compartments for a given second number of actuation contacts than would be the case with a matrix arrangement of the wiring or connection pattern of the release mechanisms.

Further, according to the first embodiment of the invention, preferably the first number is smaller than the square of the second number, and the actuation contacts are provided free of intersections, and preferably the first number is approximately given by thrice the second number less six. Furthermore, it is preferable that the first number is smaller than the square of the second number, and the actuation contacts are provided free of intersections and at one side of the compartments, preferably the first number being approximately given by twice the second number less three. With these preferred embodiments, it is advantageously possible to reduce the manufacturing costs for producing the inventive device, insofar as additional layers for providing intersections of actuation contacts can be left out. Additionally, it is possible to provide the actuation contacts only at one side of the arrangement of release mechanisms.

According to both embodiments of the invention, it is preferable that the first and/or second actuation contact comprises a selection element for selectively actuating the one compartment. Preferably, the selection element is a resistance element, wherein different compartments are selected by applying different voltages as the actuation signal. Thereby, it is possible to enhance the first number relative to the second number of actuation contacts.

According to both embodiments of the invention, it is preferable that the resistance element is a non-linear element, preferably a diode. Thereby, it is possible to select an even higher number of compartments for a given security margin and a given voltage difference.

According to the second embodiment of the invention, it is preferable that the selection element is a capacitance element or an inductance element, wherein different compartments are selected by applying different frequencies as the actuation signal. Thereby, a selection of release mechanisms by means of a selection of the frequency is very advantageously possible.

According to the second embodiment of the invention, it is preferable that the actuation contacts form a mesh-like structure in or on the substrate. It is thereby possible to achieve the advantages of the first embodiment of the present invention also for the second embodiment of the present invention.

It is preferred according to both embodiments of the present invention, that the release mechanism is a one-time release mechanism. This means that the release mechanism is “destroyed” in some manner by applying a release signal above the threshold and the release mechanism is not re-usable. Thereby, it is possible to manufacture the release mechanism in a very cost-effective and easy manner. Nevertheless, the present invention also refers to a release mechanism which is closable once it has been opened (for the first time) and re-openable at least a second time. Such an embodiment employing a re-closable and re-openable release mechanism is less preferred because it usually implies higher costs.

According to a preferred variant of both embodiments of the present invention, the release mechanism according to the present invention is provided by means of a closure cap. A closure cap is a specific and preferred embodiment of a release mechanism. Examples of other release mechanisms are: a polymer membrane or a gel that releases drugs if heated (decomposition of a carrier matrix or changing properties of it, such as breaking dedicated chemical bonds) or membranes that change their permeability for certain molecules upon the application of an electrical potential.

In a still further preferred variant of both embodiments of the present invention, a first group of compartments is provided to contain a first quantity of a first substance and a second group of compartments is provided to contain a second quantity of a second substance. An advantage of the device according to the present invention is that a very flexible substance-release mechanism can be implemented in the structure of the inventive device. For example, it is possible to provide compartments of different size, thus being capable of containing different volumes of the substance or substances to be released. For example, if at a given moment a greater quantity of a substance is to be released, a device can be controlled accordingly and open a compartment having an appropriate size and hence containing an appropriate volume of the substance to be released, instead of releasing the same quantity of a substance from a certain number of smaller compartments, which would have the same effect. Of course, the release of an appropriate quantity of a substance from one single compartment is easier to control and therefore makes the device according to the present invention smaller, lighter in weight and more cost effective. Accordingly, the first and second substance can be different or identical. Another way to improve the flexibility of releasing substances such as drugs or the like is to provide several different substances or different mixtures of substances in different compartments on the device, the different compartments being of the same or a different size. It is thereby possible to controllably release for example two different drugs alternatively during the day or during another time interval to the patient. Alternatively, it is also possible to further enhance the flexibility of use of the inventive device, for example, by providing differently sized compartments as well as different substances in the differently sized compartments. It is preferred according to the present invention, that the first quantity is approximately half of the second quantity. It is thereby also possible to have a first group of compartments having a first volume or containing a first quantity of a substance, a second group of compartments containing each twice the first quantity, a third group containing four times the first quantity and a fourth group of compartments containing eight times the first quantity. Thereby, the flexibility of releasing one or more substances is even further enhanced.

It is further preferred according to a variant of both embodiments of the present invention that the release mechanism is activated by means of an electro-chemical reaction or by means of heating the release mechanism, preferably by means of an electric current or by means of applying an electric potential between the first actuation contact and the second actuation contact. The device can be produced in a very cost-effective manner and the release of the substance can be made to take place more quickly and more accurately.

Further preferred variants of both embodiments of the present invention are provided with a control unit for controlling the release of the substance. It is further preferred that the first number is at least 10, preferably at least 100, more preferably at least 1,000, still more preferably at least 10,000 compartments. The compartments can be filled by micropipette or ink jet printing techniques.

According to a preferred variant of both embodiments of the present invention, the release mechanism of one compartment of the first number of compartments is provided so as to be power resistant up to a first power level applied without releasing the substance, and the release mechanism is provided so as to release the substance above a second power level applied to the release mechanism. Thereby, it can be assured to a very high degree that no release mechanism is actuated in cases where it should not be activated or vice versa.

It is further preferred according to a variant of both embodiments of the present invention that the first power level is approximately half the second power level or that the first power level exceeds half of the second power level. Thereby, it is advantageously possible to provide a device that releases the substance or the substances in a very reliable and controllable manner. A person skilled in the art knows that if the power applied approaches the first power level and the second power level, while the same level of reliability in releasing or not releasing the substances is maintained, this means that the resistance of the membranes has to meet higher precision requirements; nevertheless, such an approaching of the first power level and the second power level will result in the possibility to realize a more complex mesh (with a higher number of actuation contacts).

The present invention also includes a method of controllably releasing a predefined quantity of a substance from a compartment using a device comprising, according to a first embodiment of the invention, a first number of compartments in a substrate, each compartment being closed by at least one release mechanism, and the device further comprising a second number of actuating contacts, wherein the first number exceeds the second number, each of the actuation contacts comprises at least one conductor path in or on the substrate for electrically connecting at least one compartment, and the actuation contacts form a mesh-like structure in or on the substrate, the method comprising the step of:
applying an actuation signal between at least a first actuation contact and a second actuation contact of the second number of actuation contacts, thereby releasing the substance from one compartment of the first number of compartments.

The present invention also includes a method of controllably releasing a predefined quantity of a substance from a compartment using a device comprising, according to a second embodiment of the invention, a first number of compartments in a substrate, each compartment being closed by at least one release mechanism, and the device further comprising a second number of actuating contacts, wherein the first number exceeds the second number, each of the actuation contacts comprises at least one conductor path in or on the substrate for electrically connecting at least one compartment, and the one compartment is actuated dependent on the actuation signal between the first actuation contact and the second actuation contact, the method comprising the step of:
applying an actuation signal between at least a first actuation contact and a second actuation contact of the second number of actuation contacts, thereby releasing the substance from one compartment of the first number of compartments.

With both embodiments of the invention, it is possible to controllably release a specific quantity of a substance in a very rapid and easily controllable manner.

In a preferred variant of both embodiments of the method according to the present invention, more than one compartment release the substance at the same time. This may mean that a plurality of compartments are opened sequentially, such that their period of release (usually much longer than the time required for opening a specific compartment) overlap and a release of the substance by more than one compartments is possible. It is thereby possible to very flexibly control the release of a substance.

In a further preferred variant of both embodiments of the method according to the present invention, the actuation signal is adapted in voltage and/or in frequency according to the compartment to be actuated. It is thereby possible to control in a very flexible manner the release of the substance or the substances and to provide the device in a very cost-effective manner.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

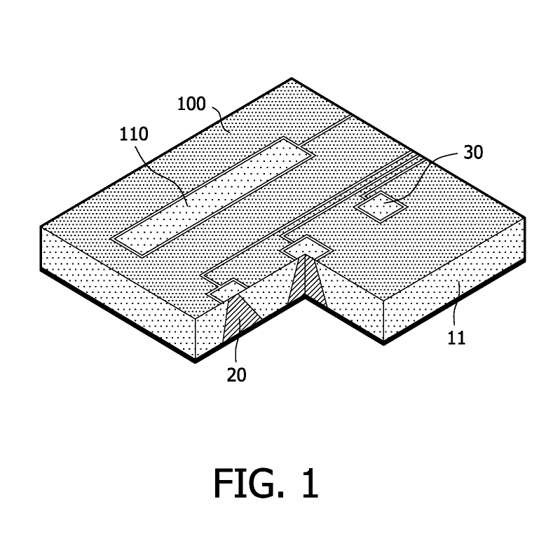

FIG. 1 illustrates schematically a device 100 according to the prior art showing a principle structure of a device of such a type.

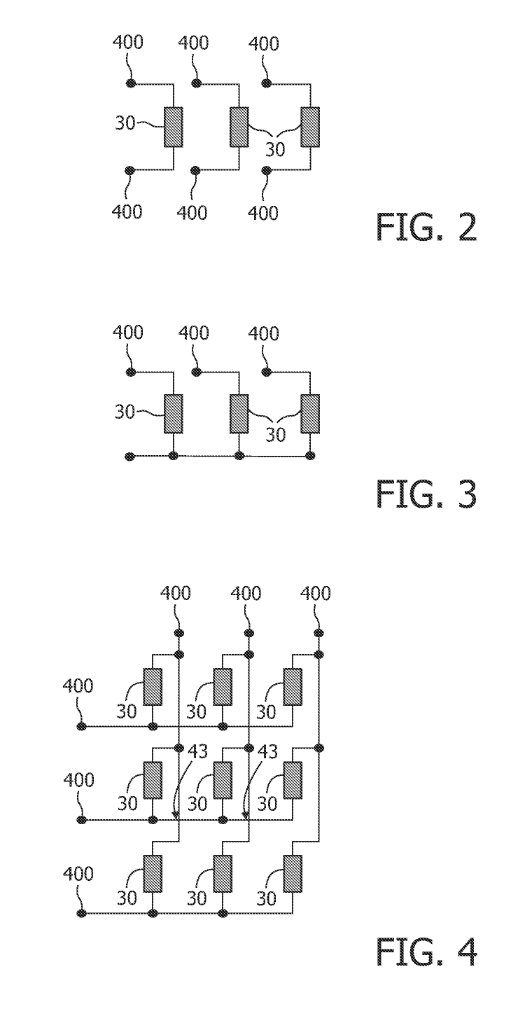

FIGS. 2 to 4 illustrate schematically a wiring pattern according to the prior art.

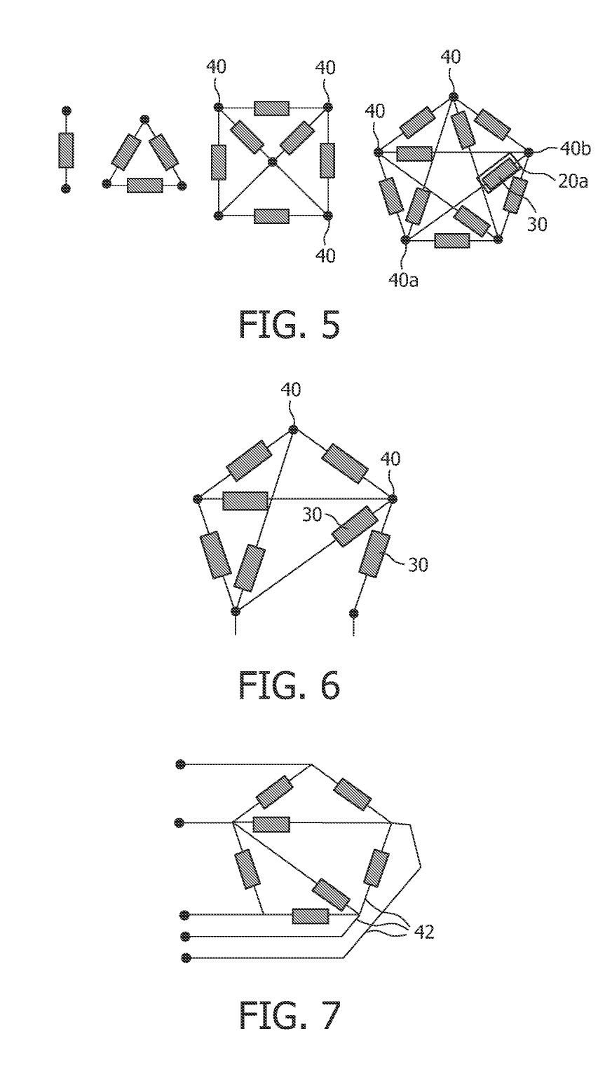

FIGS. 5 to 8 illustrate schematically different mesh-like wiring patterns according to a first embodiment of the present invention.

FIGS. 9 to 16 illustrate schematically different variants of a second embodiment of the present invention.

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1, a known device 100 according to the prior art is schematically shown. The known device 100 comprises a substrate 11 where a plurality of compartments 20 are located. The compartments 20 are closed by a release mechanism 30, especially a closure cap 30. It can further be seen from FIG. 1 that there are electrode lines going to each of the compartments 20 or at least to, or near to, each of the release mechanisms 30. The connecting lines are not denoted by means of a reference sign in FIG. 1. The known device 100 further comprises an electrode area 110.

In FIGS. 2 to 4 further wiring patterns according to the prior art are shown. FIG. 2 shows a pattern with two actuation contacts 400 provided for each compartment 20 or for each release mechanism 30. As a result, the first number N₁ would be half the second number N₂ (N₁=0,5*N₂), requiring huge wiring efforts in terms of space on the substrate and in terms of manufacturing complexity. FIG. 3 shows a pattern with one actuation contact 400 provided for each compartment 20 or for each release mechanism 30 and with a further actuation contact 400 common to a multitude of compartments 20 or release mechanisms 30. In this example, the first number N₁ would equal the second number N₂ minus one (N₁=N₂−1). FIG. 4 shows a pattern with a matrix arrangement of release mechanisms 30, i.e. the first number N₁ would equal one quarter of the square of the second number N₂ (N₁=0,25*N₂²).

FIGS. 5 to 8 show examples of the first embodiment of the present invention having mesh-like structures of actuation contacts 40. FIGS. 9 to 16 show examples and illustrations related to the second embodiment of the present invention, where release mechanisms are selected or are selectively activated depending on the actuation signal.

In both embodiments of an inventive device 10, a first number of compartments 20 or release mechanisms are present, similar to the representation in FIG. 1. The device 10 comprises the compartments 20 in a substrate 11, comparable to the prior art devices. The substrate 11 is the structural body in which the compartments 20 are formed; for example it contains the etched, machined or moulded compartments 20. A compartment 20 (which is also called a reservoir in the following) is a container for a substance. Micro-electromechanical system methods, micro-moulding and micro-machining techniques known in the art can be used to fabricate the substrate 11 together with the compartments 20 from a variety of materials. Examples of suitable substrate materials include metals, ceramics, semiconductors, degradable and non-degradable polymers. Bio-compatibility of the substrate material typically is preferred for in-vitro device applications. The substrate, or portions thereof, may be coated, capsulated, or otherwise contained in a bio-compatible material before use. The substrate 11 can be flexible or rigid. In one embodiment, the substrate 11 serves as a support for a microchip device. In one example, the substrate 11 is formed of silicon. The substrate 11 can have a variety of shapes for shaped surfaces. It can, for example, have a release side, i.e. an area having release mechanisms, that is planar or curved. The substrate may for example be in a shape selected from the group consisting of discs, cylinders, or spheres. In one embodiment, the release side can be shaped to conform to a curved tissue surface. This would be particularly advantageous for local delivery of a therapeutic agent to that tissue surface. In another embodiment, the backside (distal to the release side) is shaped to conform to an attachment surface. The substrate may consist of only one material or may be a composite or multi-laminate material, that is, composed of several layers of the same or different substrate materials that are bonded together.

FIG. 5 show examples of wiring patterns of the device 10 according to the present invention with mesh-like structures of actuation contacts 40. Leftmost, the case for N₂=3 is given (with N₁=3 compartments). In the middle of FIG. 5, the case for N₂=4 is given (with N₁=6 compartments). Rightmost, the case for N₂=5 is given (with N₁=10 compartments). In this Figure, the building principle of such mesh-like structures of actuation contacts 40 according to the present invention is visible.

This building principle consists, roughly spoken, of providing a compartment 20 or a release mechanism 30 between each of the second number N₂ of actuation contacts 40. Hereinafter, such a mesh-like structure is referred to as a fully populated mesh.

An important feature of the present invention is that it must be possible to verify that indeed the release mechanisms 30 are actuated, i.e. that the corresponding compartments 20 are indeed opened. Therefore, the device and the method according to the present invention allow to verify whether a specific release mechanism 30 has been actuated or not. In the following, it is assumed that the release mechanisms 30 are uniform, i.e. all have an equal resistance value R_{M} before they are actuated, i.e. generally destroyed or “blown”. Therefore, all these release mechanisms 30 require a specific amount of energy to be activated and have a very high resistance afterwards. Due to matching on the substrate, typical tolerances will be low, e.g. smaller than 20%. In order to allow to verify whether a specific release mechanism 30 has been actuated or not, it is necessary to define a safe power margin around the amount of energy which is needed to activate the release mechanism 30. Therefore, a first power level 61 will be defined, which is such that it is ensured that a specific release mechanism 30 considered will always stay intact if the energy conveyed to that release mechanism 30 stays below the first power level 61. A second power level 62 will be assumed to be such that it will always destroy or actuate the release mechanism 30, provided that the conveyed energy is superior to the second power level 62. For the purpose of the description of the present invention, it is assumed that the first power level 61 is half (i.e. 50%) of the second power level 62.

For the case of a fully populated mesh (cf. FIG. 5), the energy dissipated by one release mechanism 30 (corresponding to one compartment 20a) between a first actuation contact 40a and a second actuation contact 40b is V²/R_{M}, where V corresponds to the applied voltage. The voltage can now be chosen such that this energy dissipation is just enough to activate or blow the release mechanism 30 and that any other membrane or any other release mechanism 30 in the mesh will dissipate an amount of energy lower than the first power level 61, and therefore remains intact. This corresponds to the release mechanism 30 between the first and the second actuation contact 40a, 40b dissipating an amount of energy superior to the second power level 62, while all the other actuation contacts 40 dissipate an amount of energy lower than the first power level 61. An advantage of this interconnection method, i.e. one advantage of the mesh-like structure, is that the voltage V required to actuate a release mechanism 30 is the same for all membranes. The control logic (also called driver) required for such a system is therefore very simple. Such a driver only has to be able to output one specific voltage relative to the ground or leave the connection floating.

For higher values of the second number N₂, worst case situations show that the power margin becomes smaller, i.e. the first power level 61 corresponds to more than 50% of the second power level 62. The worst case situation can be calculated as follows:

The resistance between the first actuation contact 40a and the second actuation contact 40b of a fully populated mesh with N₂ actuation contacts and release mechanisms 30 with resistance value R_{M} is 2R_{M}/N₂. This resistance will never decrease when a membrane is actuated. A worst-case situation is that the release mechanisms 30 are actuated such that what is left is an actuation contact 40 with just one release mechanism 30 that connects to a fully populated mesh with N₂−1 terminals. In FIG. 6, such a situation is shown. If a power margin of at least 2 is required (i.e. the second power level 62 is twice the first power level 61), the resistance of the sub-mesh should not be smaller than R_{M}_{^{−}}*(\sqrt{2}−1)=R_{M}_{^{−}}*0,414. Therefore, the value of N₂ is limited to about 5.

However, an intelligent order, in which the release mechanisms are actuated, avoids this kind of worst-case situation. In any case, verification by means of resistance measurement becomes difficult for high values of N₂. As previously mentioned, one worst-case situation is the actuation of the first release mechanism 30. With the rest of the mesh fully intact, the resistance is 2R_{M}/N₂. When the first release mechanism 30 is activated, the resistance increases to 2R_{M}/(N₂−2). If a tolerance of 20% is assumed, the upper limit for N₂ is about 10.

In the first embodiment of the present invention, the current through the release mechanism 30 that is to be actuated is determined only by the voltage that is applied to the mesh or the wiring structure of the inventive device and of course by the resistance of the release mechanism 30. To ensure that the release mechanism 30 is quickly actuated, a negative resistance coefficient of this resistance is beneficial. When the closure cap or membrane heats up, the resistance drops and the current and therefore the dissipated power will increase, thus accelerating the increase in temperature further. A negative resistance coefficient will therefore increase the power margin.

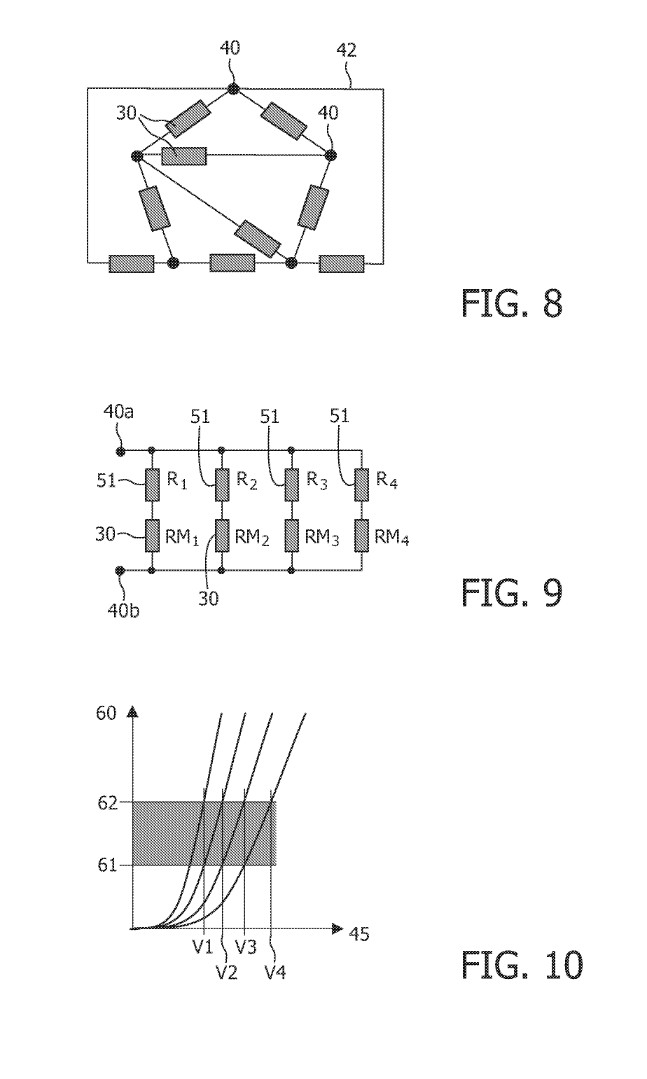

In FIGS. 7 and 8, further embodiments of a mesh-like structure of the wiring of the actuation contacts 40 are shown. A fully populated mesh with N₂>4 contains crossings or intersections of the conductor pads of different actuation contacts 40, which translates into the necessity of wires in a physical implementation of the wiring structure. This may be costly to implement in an actual product. Therefore, it is proposed according to a variant of the first embodiment of the present invention to construct a mesh without intersections or without vias, i.e. a mesh which is not fully populated. If we assume that the connections are at one side of the substrate, the maximum number of release mechanisms 30 is limited to 2N₂−3. For N₂>5, the number of release mechanisms 30 that can be connected without crossings or without intersections is considerably smaller than with crossings. Still, the number without crossings or intersections is about twice as high as in the case of the straightforward, separately addressable release mechanisms 30 with a common electrode (cf. FIG. 3), for which the number of release mechanisms 30 is N₂−1.

In FIG. 8, a further variant of the first embodiment of the present invention is shown, where the actuation contact 40 consists of pads or connecting pads that can be placed at an arbitrary point on the substrates, i.e. wires or contact pads of other actuation contacts 40 can go around the actuation contacts 40 and the maximum number of release mechanisms 30 is now 3N₂−6, which is shown in FIG. 8 for the example N₂=5.

In FIGS. 9 to 16, variants of a second embodiment of the present invention are shown. In the second embodiment of the present invention, additional resistors are inserted into the wiring structure of the inventive device. For a certain number of release mechanisms 30, two specific actuation contacts 40a, 40b are provided. By using additional resistors 51 as selection elements 51 (cf. FIG. 9), it is possible to select the different release mechanisms to be activated by means of applying different voltages V₁, V₂, V₃, V₄ (cf FIG. 10, which shows (besides the first power level 61 and the second power level 62) the dissipated power 60 as a function of the actuation signal 45 for the different release mechanisms 30 and with different selection elements 51. When applying a voltage V across the terminals 40a, 40b, i.e. the first and second actuation contact 40a, 40b, the dissipation of a release mechanism 30 is defined by V₂*R_{Ml}/(R_{Mi}+Rᵢ)*(R_{Mi}+Rᵢ). By selecting proper values, for example, if four release mechanisms 30 are connected as shown in FIG. 9, we can choose Rₗ=0 Ohm, causing R_{Ml} to be actuated at Vₗ. A margin of 2 between the power dissipated in the release mechanism 30 to be activated and the next release mechanism 30 that should not be actuated is achieved by choosing R₂=R_{M} (\sqrt{2}−1)=R_{M}*0414. V₂ is then \sqrt{2}*V₁. Similarly, R₃=R_{M}*(\sqrt{4}−1)=R_{M} resulting in V₃=2 V₁. Finally, R₄=R_{M}*(\sqrt{8}−1)=R_{M}_{^{−}}*1,828 causes V₄=2\sqrt{2}*V₁. This means that with a voltage range of only 2.82, already four membranes can be individually controlled with the first and the second actuation contact 40a, 40b by means of applying different voltages V₁, V₂, V₃ and V₄. For a higher number of membranes, the dissipation of the last serial resistor and the activation voltage will probably become too high. The temperature coefficient of the resistors and of the release mechanisms 30 in this configuration should preferably be close to 0 to maintain a well-defined voltage for actuating a release mechanism 30.

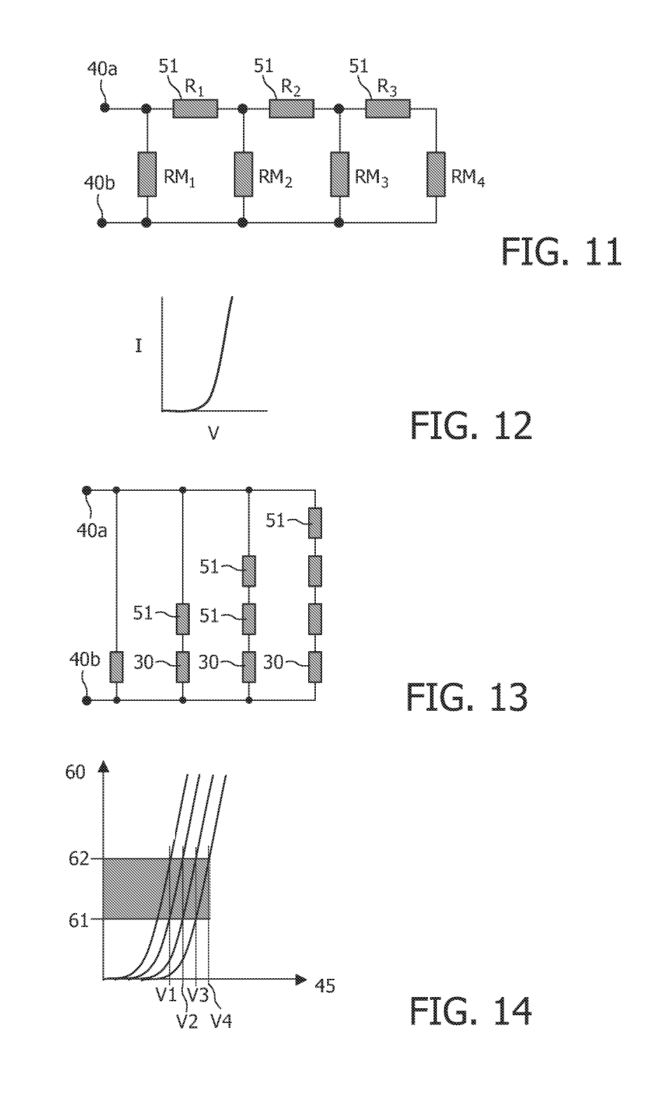

The additional resistors 51 used as selection elements 51 can also be placed in an alternative arrangement, as is shown in FIG. 11. A disadvantage in comparison with the arrangement of FIG. 9 is that the power dissipated in a release mechanism 30 that is not to be actuated changes at the moment of (?) actuation of another membrane.

The additional resistors 51 are preferably made using the same process and the same materials as the resistors of the release mechanisms 30. This enables very good matching between these two. Of course, they must be made in such a way that they can easily withstand the power they would have to dissipate. Especially the last additional resistor in the ladder will have a rather high dissipation. Again, verification of the status of the membranes (i.e. release mechanisms 30) can be done by means of a simple current or resistance measurement.

Furthermore, according to a variant of the second embodiment of the present invention, it is also possible that non-linear elements are provided as selection elements 51. The resulting circuits can be connected in parallel. This is shown in FIG. 13. The behavior of one of such non-linear resistor elements is roughly shown in FIG. 12, where the behavior of the current I versus the tension V is schematically shown (without any units). The current through a release mechanism 30 now non-linearly depends on the voltage at the actuation contacts 40a, 40b. This increases the margin between the programming voltages per membrane as compared to the use of linear resistors. Various types of non-linear elements could be used, such as non-linear resistors or diodes. Non-linear resistors can be made using low-cost deposition processing, which fits well in the production process of the device of the present invention. For instance, by connecting two different metal layers, Schottky-diodes can be formed.

By inserting a non-linearly behaving selection element 51 in the path of a release mechanism 30, a threshold value is effectively set below which there hardly flows any current. By inserting two or more such resistors in the path, each membrane or each release mechanism 30 can have an individual threshold voltage as shown in FIG. 14. The result is that the various activation voltages span a smaller range. Furthermore, they are more evenly spaced over such a voltage range. This allows a higher number of membranes to be connected to the first and the second actuation contact 40a, 40b. This behavior is shown in FIG. 14 where again the first power level 61 and the second power level 62 are shown as well as the dissipated power 60 as a function of the actuation signal 45 applied between the first and the second actuation contact 40a, 40b.

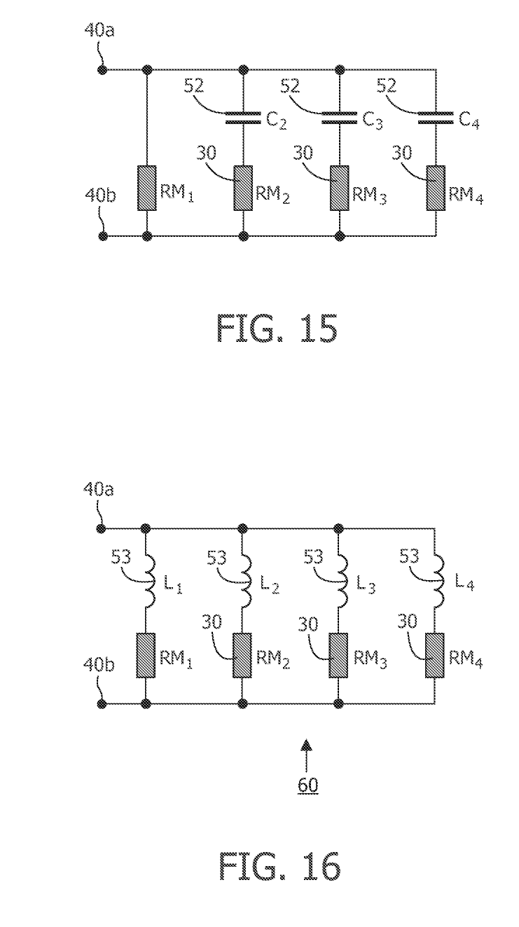

In a further variant of the second embodiment of the present invention, capacitive selection elements 52 (cf. FIG. 15) or inductive selection elements 53 (cf. FIG. 16) are schematically shown. The selection of the proper release mechanism 30 is not based on different voltages in the actuation signal 45 but on different frequencies. Serial capacitors make the dissipation in the release mechanism 30 dependent on the frequency of the actuation signal 45. Impedance measurement as a function of the frequency allows verification of the status of the membranes. The frequencies to be used are fairly high in order to limit the manufacturing costs for the different capacitances. Instead of capacitors, inductors can also be used as selection elements.

1. Device (10) for the controlled release of a predefined quantity of a substance, the device (10) comprising a first number (N1) of compartments (20) in a substrate (11), each compartment (20) being closed by at least one release mechanism (30), and the device (10) further comprising a second number (N2) of actuating contacts (40) for the release of the substance from one compartment (20a) of the first number (N1) of compartments (20) by means of applying an actuation signal (45) between at least a first actuation contact (40a) and a second actuation contact (40b) of the second number (N2) of actuation contacts (40), wherein the first number (N1) exceeds the second number (N2), and each of the actuation contacts (40) comprises at least one conductor path (42) in or on the substrate (11) for electrically connecting at least one compartment (20a), and the actuation contacts (40) form a mesh-like structure in or on the substrate (11).

2. Device (10) for the controlled release of a predefined quantity of a substance, the device (10) comprising a first number (N1) of compartments (20) in a substrate (11), each compartment (20) being closed by at least one release mechanism (30), and the device (10) further comprising a second number (N2) of actuating contacts (40) for the release of the substance from one compartment (20a) of the first number (N1) of compartments (20) by means of applying an actuation signal (45) between at least a first actuation contact (40a) and a second actuation contact (40b) of the second number (N2) of actuation contacts (40), wherein the first number (N1) exceeds the second number (N2), and each of the actuation contacts (40) comprises at least one conductor path (42) in or on the substrate (11) for electrically connecting at least one compartment (20a), and the one compartment (20a) is actuated dependent on the actuation signal (45) between the first actuation contact (40a) and the second actuation contact (40b).

3. Device (10) according to claim 1, wherein the first number (N1) exceeds one quarter of the square of the second number (N2), and preferably the first number (N1) is approximately given by one half of the square of the second number (N2) less one half of the second number (N2).

4. Device (10) according to claim 1, wherein the first number (N1) is smaller than the square of the second number (N2), and the actuation contacts (40) are provided free of intersections, and preferably the first number (N1) is approximately given by thrice the second number (N2) less six.

5. Device (10) according to claim 4, wherein the first number (N1) is smaller than the square of the second number (N2), and the actuation contacts (40) are provided free of intersections and at one side of the compartments (20), preferably the first number (N1) being approximately given by twice the second number (N2) less three.

6. Device (10) according to claim 1, wherein the first and/or second actuation contact (40a, 40b) comprises a selection element (51, 52, 53) for selectively actuating the one compartment (20a).

7. Device (10) according to claim 6, wherein the selection element (51, 52, 53) is a resistance element (51), and different compartments (20a, 20b) are selected by applying different voltages as the actuation signal (45).

8. Device (10) according to claim 7, wherein the resistance element (51) is a non-linear element, preferably a diode.

9. Device (10) according to claim 6, wherein the selection element (51, 52, 53) is a capacitance element (52) or an inductance element (53), and different compartments (20a, 20b) are selected by applying different frequencies as the actuation signal (45).

10. Device (10) according to claim 2, wherein the actuation contacts (40) form a mesh-like structure in or on the substrate (11).

11. Device (10) according to claim 1, wherein the release mechanism (30) is a one-time release mechanism (30).

12. Device (10) according to claim 1, wherein the release mechanism (30) is a closure cap.

13. Device according to claim 1, wherein the release mechanism (30) is activated by heating the release mechanism (30).

14. Device (10) according to claim 1, wherein the first number (N1) is at least 10, preferably at least 100, more preferably at least 1,000, still more preferably at least 10,000.

15. Device (10) according to claim 1, wherein the release mechanism (30) of one compartment (20a) of the first number (N1) of compartments (20) is provided so as to be power resistant up to a first power level (61) applied without releasing the substance, and the release mechanism (30) is provided so as to release the substance above a second power level (62) applied to the release mechanism (30).

16. Device (10) according to claim 15, wherein the first power level (61) is approximately half the second power level (62) or wherein the first power level (61) exceeds half of the second power level (62).

17. Method of controllably releasing a predefined quantity of a substance from at least a compartment (20) using a device (10) comprising a first number (N1) of compartments (20) in a substrate (11), each compartment (20) being closed by at least one release mechanism (30), and the device (10) further comprising a second number (N2) of actuating contacts (40), wherein the first number (N1) exceeds the second number (N2), each of the actuation contacts (40) comprises at least one conductor path (42) in or on the substrate (11) for electrically connecting at least one compartment (20a), and the actuation contacts (40) form a mesh-like structure in or on the substrate (11), the method comprising the step of:
applying an actuation signal (45) between at least a first actuation contact (40a) and a second actuation contact (40b) of the second number (N2) of actuation contacts (40), thereby releasing the substance from one compartment (20a) of the first number (N1) of compartments (20).

18. Method of controllably releasing a predefined quantity of a substance from at least a compartment (20) using a device (10) comprising a first number (N1) of compartments (20) in a substrate (11), each compartment (20) being closed by at least one release mechanism (30), and the device (10) further comprising a second number (N2) of actuating contacts (40), wherein the first number (N1) exceeds the second number (N2), each of the actuation contacts (40) comprises at least one conductor path (42) in or on the substrate (11) for electrically connecting at least one compartment (20a), and the one compartment (20a) is actuated dependent on the actuation signal (45) between the first actuation contact (40a) and the second actuation contact (40b), the method comprising the step of:
applying an actuation signal (45) between at least a first actuation contact (40a) and a second actuation contact (40b) of the second number (N2) of actuation contacts (40), thereby releasing the substance from one compartment (20a) of the first number (N1) of compartments (20).

19. Method according to claim 18, wherein the actuation signal (45) is adapted in voltage and/or in frequency according to the compartment (20a) to be actuated.
